(19) United States
(12) Patent Application Publication
(10) Pub. No.: US 2024/0361292 A1
(43) Pub. Date: Oct. 31, 2024

(54) METHOD FOR ASSESSING RIPENESS OF FRUIT AND SYSTEM FOR ASSESSING RIPENESS OF FRUIT

(71) Applicant: Panasonic Intellectual Property Management Co., Ltd., Osaka (JP)

(72) Inventors: Yoshiki YAMADA, Osaka (JP); Yosuke HANAI, Osaka (JP)

(21) Appl. No.: 18/687,209

(22) PCT Filed: Aug. 26, 2022
(86) PCT No.: PCT/JP2022/032241

§ 371 Date: Feb. 27, 2024

(30) Foreign Application Priority Data
Aug. 31, 2021 (JP) 2021-141820

(51) Int. Cl. G01N 33/02

(52) U.S. Cl. CPC G01N 33/025

(57) ABSTRACT

A volatile component released from an epicarp of a banana or an avocado is collected. By using a detector, a detection result of the volatile component is obtained. The detector is configured to output the detection result in accordance with an amount of one or more marker components selected from the group consisting of specific compounds. Based on the detection result, the ripeness of the banana or the avocado is assessed.

### TECHNICAL FIELD

The present disclosure relates to methods for assessing ripeness of fruit and systems for assessing ripeness of fruit and specifically relates to a method for assessing ripeness of fruit on the basis of a gas released from the fruit and a system for assessing ripeness of fruit, the system being configured to implement the method.

### BACKGROUND ART

Patent Literature 1 discloses that avocados grown to a prescribed size are harvested from trees in a production area, the avocados thus harvested are transported from the production area to a processing plant, the avocados thus transported are afterripened by warming in the processing plant and are then offered to consumers, wherein a harvest timing is selected such that the avocados are harvested after parts of skins of the avocados turn black.

### CITATION LIST

### Patent Literature

Patent Literature 1: JP 2002-330625 A

### SUMMARY OF INVENTION

It is an object of the present disclosure to provide a method for assessing ripeness of fruit and a system for assessing ripeness of fruit, the method and the system being capable of assessing ripeness of bananas or avocados.

A method for assessing ripeness of fruit according to an aspect of the present disclosure is a fruit ripeness assessing method. The fruit is bananas. A volatile component released from an epicarp of the fruit is collected. A detection result of the volatile component is acquired by using a detector. The detector is a means for outputting the detection result in accordance with an amount of one or more marker components selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate. The ripeness of the fruit is assessed based on the detection result.

A method for assessing ripeness of fruit according to an aspect of the present disclosure is a fruit ripeness assessing method. The fruit is avocados. A volatile component released from an epicarp of the fruit is collected. A detection result of the volatile component is acquired by using a detector. The detector is a means for outputting the detection result in accordance with an amount of one or more marker components selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone. The ripeness of the fruit is assessed based on the detection result.

A system for assessing ripeness of fruit according to an aspect of the present disclosure is a system implementing the method for assessing ripeness of fruit. The system includes the detector and an assessing member configured to assess the ripeness of the fruit on a basis of the detection result output from the detector.

### BRIEF DESCRIPTION OF DRAWINGS

FIG. 1 is a schematic system configuration diagram of a sensor device and a system for assessing ripeness according to an embodiment of the present disclosure;

FIG. 2 is a schematic illustrative view of a gas sensor included in the sensor device;

FIG. 3A is a graph for bananas, the graph showing a relationship between the number of days elapsed and a color (HUE value) of an epicarp;

FIG. 3B is a graph for the bananas, the graph showing a relationship between the number of days elapsed and hardness of an mesocarp;

FIG. 4 is a graph for the bananas, the graph showing a relationship between the color (HUE value) of the epicarp and the hardness of the mesocarp;

FIG. 5A is a graph for the bananas, the graph showing a relationship between a result of assessing the color of the epicarp from a detection result of a volatile component by using a gas chromatograph and an actual color of the epicarp;

FIG. 5B is a graph for the bananas, the graph showing a relationship between a result of assessing the hardness of the mesocarp from the detection result of the volatile component by using the gas chromatograph and actual hardness of the mesocarp;

FIG. 6A is a graph for the bananas, the graph showing a relationship between a result of assessing the color of the epicarp from a detection result of a volatile component by using a sensor device and the actual color of the epicarp;

FIG. 6B is a graph for the bananas, the graph showing a relationship between a result of assessing the hardness of the mesocarp from the detection result of the volatile component by using the sensor device and actual hardness of the mesocarp;

FIG. 7A is a graph for the bananas, the graph showing a relationship between the number of days elapsed and a peak strength of isopentyl alcohol included in the detection result of the volatile component by using the gas chromatograph;

FIG. 7B is a graph showing a relationship between the number of days elapsed and a peak strength of 1-methoxy-2-propanol included in the detection result of the volatile component by using the gas chromatograph;

FIG. 7C is a graph showing a relationship between the number of days elapsed and a peak strength of 1-butanol included in the detection result of the volatile component by using the gas chromatograph;

FIG. 8A is a graph for avocados, the graph showing a relationship between the number of days elapsed and a color (HUE value) of a mesocarp;

FIG. 8B is a graph for the avocados, the graph showing a relationship between the number of days elapsed and hardness of the mesocarp;

FIG. 9 is a graph for the avocados, the graph showing a relationship between the color (HUE value) of the mesocarp and the hardness of the mesocarp;

FIG. 10A is a graph for the avocados, the graph showing a relationship between a result of assessing the color of the mesocarp from a detection result of a volatile component by using a gas chromatograph and an actual color of the mesocarp;

FIG. 10B is a graph for the avocados, the graph showing a relationship between a result of assessing the hardness of the mesocarp from the detection result of the volatile component by using the gas chromatograph and actual hardness of the mesocarp;

FIG. 11A is a graph for the avocados, the graph showing a relationship between a result of assessing a color of the mesocarp from a detection result of the volatile component by using a sensor device and the actual color of the mesocarp; and

FIG. 11B is a graph for the avocados, the graph showing a relationship between a result of assessing the hardness of the mesocarp from the detection result of the volatile component by using the sensor device and the actual hardness of the mesocarp;

### DESCRIPTION OF EMBODIMENTS

An embodiment of the present disclosure will be described below.

First of all, how the inventors developed the present disclosure will be briefly described.

Fruit, such as bananas and avocados, ripens with time also after harvested and thus has to be managed such that the fruit ripened to an appropriate degree is offered for sale in retail establishments.

With the technology described in Patent Literature 1 (JP 2002-330625 A), a harvest timing is selected based on the color of skins of avocados, but it is difficult to check the degree of ripening thereafter.

Bananas and avocados temporarily release ethylene in an early stage of ripening to promote their ripening by the action of the ethylene as a hormone. The inventors considered evaluating the ripeness of the bananas and avocados by quantifying the ethylene. However, a timing at which the ethylene is released is limited, and at other timings, the ripeness cannot be evaluated based on the ethylene.

Moreover, regarding bananas, most bananas to be consumed in Japan are imported from their countries of origin, and the Plant Protection Act prohibits the import of ripe yellow bananas to prevent pest infestation. Therefore, unripe green bananas are harvested in countries of origin, and are then imported to Japan. The bananas arrived at Japan are ripened by subjecting to treatment called afterripening before they are on sale in retail establishments. The afterripening is performed by subjecting the bananas to an ethylene gas, for example, in a room (called “Muro” in Japanese). A condition for the afterripening influences sweetness and flavor of the bananas.

Storing green bananas imported to Japan at an ordinary temperature for two to three weeks before the afterripening makes almost no change in the hardness and appearance of the bananas, but according to independent study by the inventors, the longer a storage period of the green bananas are, the riper the bananas are due to the afterripening. This means that even though the bananas are green, the bananas ripen with time, which may change an optimal condition for the afterripening of the bananas.

Almost no change is, however, made in the hardness and appearance of epicarps of the green bananas during the storage as explained above, and therefore, the ripeness of the bananas is difficult to be checked based on the hardness and appearance of the epicarps of the bananas. The ripeness of the bananas may be checked by checking the hardness of mesocarps of the bananas, but in that case, the epicarps of the bananas have to be removed, thereby reducing product values of the bananas. The hardness of the mesocarps may be checked by strongly pressing the epicarps of the bananas, but the mesocarps tend to be damaged, which thus also reduces the product values of the bananas. Moreover, the green bananas release almost no ethylene, and therefore, the ripeness is difficult to be evaluated based on the ethylene. Thus, setting a condition for the afterripening in accordance with the ripeness of the green bananas is very difficult, and therefore, placing appropriately ripe bananas on sale in retail establishments is not easy.

Moreover, regarding avocados, the hardness and appearance of epicarps of the avocados hardly change even when the avocados ripen, and therefore, assessing the ripeness of the avocados on the basis of the hardness and appearance of the epicarps is also difficult. Also in the case of the avocados, the hardness of mesocarps may be checked to check the ripeness, but in that case, product values of the avocados are reduced similarly to the case of the bananas.

In view of the problems, the inventors independently conducted study, and as a result, found that each of bananas and avocados during the storage releases a gas which is specific and which is other than ethylene and that the release amount of the gas changes with time. The inventors have developed a method for detecting the gas released from each of the bananas and the avocados and assessing the ripeness on the basis of a result of the detection.

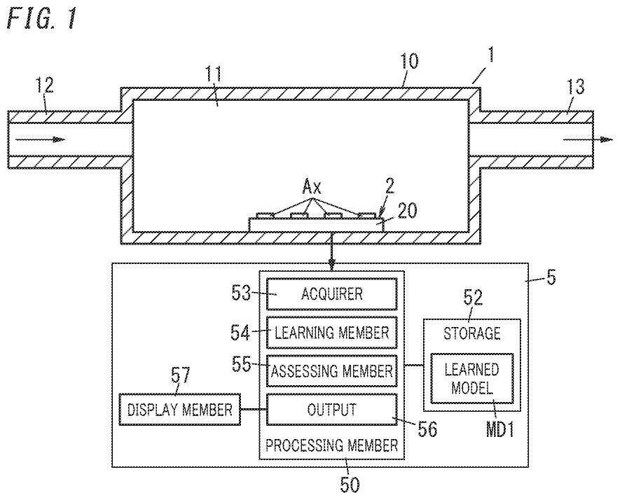

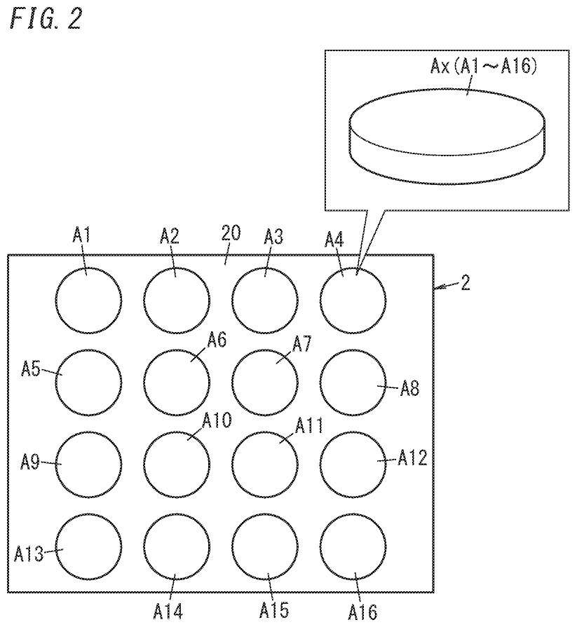

An embodiment and variations will be described with reference to FIGS. 1 and 2. Note that the embodiment and variations described below are mere examples of various embodiments of the present disclosure. Moreover, the embodiment and variations described below may be modified variously depending on design and the like as long as the object of the present disclosure is achieved. Moreover, configurations of the variations may accordingly be combined.

The drawings below are schematic views, and the dimensional ratio of components in the figures is not necessarily reflect the actual dimensional ratio.

A method for assessing ripeness of fruit according to the embodiment of the present disclosure is a fruit ripeness assessing method. The fruit is bananas or avocados.

When the fruit is bananas, a volatile component released from an epicarp of the fruit is collected in the method. By using a detector (output means) 1, a detection result of the volatile component is obtained. The detector 1 in this case is a means for outputting the detection result in accordance with an amount of one or more marker components selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate. Based on the detection result, the ripeness of the fruit is assessed.

A banana releases 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate, and release amounts of these components change along with the ripeness of the banana. Therefore, at least one of these components is used as a marker component, and a detection result according to an amount of the marker component released from the banana is obtained, and thereby, the ripeness of the banana can be assessed based on the detection result.

When the fruit is avocados, a volatile component released from an epicarp of the fruit is collected in the method. By using a detector 1, a detection result of the volatile component is obtained. The detector 1 in this case is a means for outputting the detection result in accordance with an amount of one or more marker components selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone. Based on the detection result, the ripeness of fruit is assessed.

An avocado releases 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone, and release amounts of these components change along with the ripeness of the avocado. Therefore, at least one of these components is used as a marker component, and a detection result according to an amount of the marker component released from the avocado is obtained, and thereby, the ripeness of the avocado can be assessed based on the detection result.

The present embodiment will be described in further detail.

In the present embodiment, bananas are a type of fruit that is in the genus Musa of the family Musaceae and that has an edible mesocarp. Typical product types of the bananas are, for example, Giant Cavendish, Taiwanese banana, Banapple, Lakatan, senorita banana, Ryukyu banana, Dwarf Cavendish banana, Morade, and Plantain. Avocados are a type of fruit that comes from evergreen trees in the genus Persea of the family Lauraceae and that has an edible mesocarp. Typical product types of the avocados are, for example, Haas, Fuerte, Bacon, Pinkerton, and Reed.

The detection result output from the detector 1 is information depending on the amount of the marker component in the volatile component, and as long as this is the case, the detection result may be information directly representing the amount of the marker component or may be information which does not directly represent the amount of the marker component.

The detector 1 is preferably a means for outputting the detection result according to amounts of two or more marker components. That is, when the fruit is bananas, the detector 1 is preferably a means for outputting the detection result in accordance with the amounts of two or more marker components selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate. When the fruit is avocados, the detector 1 is preferably a means for outputting the detection result in accordance with the amounts of two or more marker components selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone. The detection result may be a collection of two or more pieces of information and the like corresponding to respective amounts of the two or more marker components, or may be information which depends on the amount of each of the two or more marker components but which is not separated into pieces of information corresponding to the respective amounts of the two or more marker components.

In this case, using the two or more marker components enables the ripeness of the fruit to be more accurately assessed. In particular, assessing the ripeness of the fruit by not only individually using the amounts of the two or more marker components but also using a mutual relationship between the amounts of the two or more marker components enables the ripeness of the fruit to be more accurately assessed.

The detector 1 is not particularly limited as long as it outputs the detection result according to the amount(s) of one, or two or more, marker component(s). Aspects of the detection result are not limited as long as the detection result is a result depending on the amount(s) of the marker component(s). For example, the detection result may be a numerical value or may be a pattern such as a waveform.

The detector 1 includes, for example, a gas sensor 2. In this case, for example, a signal output from the gas sensor 2 when the volatile component is supplied to the gas sensor 2 or information obtained by converting the signal is the detection result.

When the detector 1 includes the gas sensor 2, the gas sensor 2 may be a sensor array including plurality of sensor elements Ax having different sensory characteristics. In this case, the detection result is, for example, a collection of signals output from the plurality of sensor elements Ax or pieces of information obtained by converting the signals. When the gas sensor 2 is a sensor array as in this case, the ripeness can be assessed from combinations of the plurality of pieces of information, thereby increasing assessment accuracy of the ripeness.

FIG. 1 shows an example of the sensor device which is the detector 1 including the gas sensor 2. FIG. 1 also shows a ripeness assessing system 5 including the detector 1, but the ripeness assessing system 5 will be described later, and the sensor device will be described at first.

The sensor device includes a sensor room 10, the gas sensor 2, and a substrate 20.

The sensor room 10 has an accommodation space 11 therein. To the sensor room 10, an introduction path 12 and an exhaust path 13 each communicated with the accommodation space 11 are connected. The sensor room 10 is configured such that the volatile component is introduced through the introduction path 12 into the accommodation space 11 and the volatile component in the accommodation space 11 is further discharged from the accommodation space 11 through the exhaust path 13 to the outside. The sensor device may include an air blower and the like for sending a sample gas into the accommodation space 11. The gas sensor 2 and the substrate 20 are accommodated in the accommodation space 11. In the accommodation space 11, the substrate 20 is disposed, and on the substrate 20, the gas sensor 2 is disposed.

The gas sensor 2 outputs a signal according to the amount of the marker component as described above. For example, the gas sensor 2 changes the electrical characteristic value thereof in response to the marker component, and the amount of change in the electrical characteristic value depends on the amount of the marker component.

In the present embodiment, the gas sensor 2 is the sensor array including the plurality of sensor elements Ax having different sensory characteristics. In the present embodiment, the gas sensor 2 includes 16 sensor elements Ax. The 16 sensor elements Ax may be denoted as sensor elements A1 to A16 (see FIG. 2). The 16 sensor elements A1 to A16 are disposed in four rows and four columns on the substrate 20.

Each of the plurality of sensor elements Ax includes, for example, a matrix including an organic material and electrically conductive particles dispersed in the matrix. Each sensor element Ax shown in FIG. 2 is a film having a circular shape in plan view, but the shape of each sensor element Ax is not limited to this example.

As the organic material, a material having the property of adsorbing the marker component is selected. The organic material includes, for example, at least one selected from the group consisting of adipic acid polydiethylene glycol, succinic acid diethylene glycol, diglycerol, tetrahydroxy ethylene diamine, poly(ethylene glycol succinate), polyethylene glycol 4000 (manufactured by Sigma-Aldrich Co. LLC), polyethylene glycol 20000 (manufactured by Sigma-Aldrich Co. LLC), polyethylene glycol 20M (manufactured by Shinwa Chemical Industries Ltd.), free fatty acid phase (Free Fatty Acid Polymer, manufactured by Shinwa Chemical Industries Ltd.), 1,2,3-tris (2-cyanoethoxy) propane, N,N-bis(2-cyanoethyl) formamide, Lac-3R-728 (manufactured by GL Sciences Inc.), Reoplex 400 (manufactured by Shinwa Chemical Industries Ltd.), SP-2330 (manufactured by Sigma-Aldrich Co. LLC), SP-2340 (manufactured by Sigma-Aldrich Co. LLC), and UCON 75-HB-90000 (manufactured by Shinwa Chemical Industries Ltd.). Each of these materials has the property of adsorbing at least one component selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate and is thus usable for assessing the ripeness of bananas. Moreover, each of these materials has the property of adsorbing at least one component selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone and is thus usable for assessing the ripeness of avocados.

When the gas sensor 2 includes the plurality of sensor elements Ax, if the plurality of sensor elements Ax includes different organic materials, the plurality of sensor elements Ax can have different sensory characteristics. The organic material is not limited to those explained above as long as it has the property of adsorbing the marker component.

The electrically conductive particles include, for example, at least one material selected from the group consisting of a carbon material, an electrically conductive polymer, metal, metal oxide, a semiconductor, a superconductor, and a complex compound. The carbon material includes, for example, at least one material selected from the group consisting of carbon black, graphite, coke, carbon nanotube, graphene, and fullerene. The electrically conductive polymer includes, for example, at least one material selected from the group consisting of polyaniline, polythiophene, polypyrrole, and polyacetylene. The metal includes, for example, at least one material selected from the group consisting of silver, gold, copper, platinum, and aluminum. The metal oxide includes, for example, at least one material selected from the group consisting of indium oxide, tin oxide, tungsten oxide, zinc oxide, and titanium oxide. The semiconductor includes, for example, at least one material selected from the group consisting of silicon, gallium arsenide, indium phosphide, and molybdenum sulfide. The superconductor includes, for example, at least one material selected from the group consisting of YBa₂Cu₃O₇ and Tl₂Ba₂Ca₂Cu₃O₁₀. The complex compound includes, for example, at least one material selected from the group consisting of a complex compound of tetra methyl paraphenylene diamine and Chloranil, a complex compound of tetra cyanoquinodimethane and alkali metal, a complex compound of tetrathiafulvalene and halogen, a complex compound of iridium and a halocarbonyl compound, and tetracyano platinum.

When the organic material in each sensor element Ax adsorbs the marker component, the volume of the matrix increases, thereby increasing the distance between the electrically conductive particles in each sensor element Ax. Accordingly, the electrical resistance value of each sensor element Ax increases. As the amount of the marker component adsorbed on the organic material increases, the electrical resistance value of each sensor element Ax increases. Thus, a change in the electrical resistance value of each sensor element Ax is information depending on the amount of the marker component.

The substrate 20 includes an electrode connected to each sensor element Ax. When a voltage is applied via the electrode to each sensor element Ax, a current according to the electrical resistance value of each sensor element Ax flows through the sensor element Ax. The current according to the electrical resistance value or information obtained by converting the current is obtained as an output of each sensor element Ax. A collection of outputs of the sensor elements Ax is the detection result by the sensor device.

The detector 1 may be a gas chromatograph. In that case, for example, when the volatile component is supplied to the gas chromatograph, a chromatogram output from the gas chromatograph or information obtained by converting the chromatogram is the detection result.

The detector 1 may be any means other than that explained above.

Aspects of the detector 1 when the detector 1 includes the gas sensor are not limited to the example above. For example, the aspect of the gas sensor is not limited to the example above, and, for example, when the marker component is adsorbed on, is connected to, is trapped by, or interacts with, an appropriate gas sensor, for example, the intensity of, or the amount of change in, the weight, electrical characteristics (electrical resistance value, permittivity, and the like), resonance frequency, quantity of light emitted, or amount of radiation radiated of the gas sensor may be obtained as the detection result.

The detector 1 may be a means that liquefies or solidifies the marker component in the volatile component by condensation or the like and then measures the weight of the marker component.

The detector 1 may be a means that measures the absorbance of the marker component in the volatile component to quantify the marker component.

The detector 1 may be a means that outputs, as the detection result, a signal obtained from the gas detector when the volatile component is introduced into a measuring apparatus including the gas detector directly or in a state where the volatile component is held by an adsorption tube. The measuring apparatus may be provided, upstream of the detector, with a separation device such as a capillary column for separating the marker component from the volatile component. An example of the detector 1 in this case is a gas chromatograph. The detector is, for example, a detector using catalytic oxidation non-dispersive infrared absorption (an NDIR method), a detector using hydrogen flame ionization detection (a FID method), a photo ionization detector (PID), or a mass spectrometer (MS), or a semiconductor gas sensor.

The detector 1 may be a detecting tube. The detecting tube is, for example, a glass tube which is densely filled with a detection agent reactive to the marker component and which has a surface with a scale. When the volatile component is introduced into the detecting tube, part of the detection agent which has reacted with the marker component discolors. The degree of discoloring of the detection agent is the detection result. For example, the length of a discolored part of the detection agent is read based on the scale, and from the length, the amount of the marker component introduced into the detecting tube can be quantified.

To assess the ripeness on the basis of the detection result, executing an arithmetic process on the detection result enables an evaluation to be carried out. To execute the arithmetic process, executing an arithmetic process using an evaluation model enables the evaluation on the basis of the detection result to be carried out. In this case, the evaluation model may be a learned model obtained by machine learning using learning data. For example, combinations of detection results and ripeness of fruit whose ripeness is known are accumulated as the learning data in advance. The learning data is used to create a learned model for assessing the ripeness from the detection results. In this way, using the learned model enables the ripeness to be assessed from the detection results of the fruit whose ripeness is unknown. To create the learned model, for example, a program (algorithm) of artificial intelligence is caused to create the learned model by machine learning of the learning data. The program of the artificial intelligence is a model of the machine learning and is, for example, a random forest method or a neural network.

The ripeness assessing system 5 for fruit will be described. The ripeness assessing system 5 for fruit implements a method for assessing ripeness of bananas or avocados which are fruit. The ripeness assessing system 5 includes the detector 1 and an assessing member 55 configured to assess the ripeness of fruit on the basis of the detection result output from the detector 1.

FIG. 1 shows an outline of a configuration example of the ripeness assessing system 5 including the sensor device as the detector 1. Note that as explained above, the detector 1 is not limited to the sensor device.

In the example shown in FIG. 1, the ripeness assessing system 5 includes: a processing member 50 including the assessing member 55; a storage 52; and a display member 57.

The processing member 50 is a control circuit that controls operation of the ripeness assessing system 5. The processing member 50 can be implemented by, for example, a computer system including one or more processors (microprocessors) and one or more memory elements. That is, the one or more processors execute one or more programs (applications) stored in the one or more memory elements, thereby functioning as the processing member 50. Here, the program(s) is stored in the memory element(s) or the storage 52 of the processing member 50 in advance, but the program(s) may be provided over a telecommunications network such as the Internet or may be provided as a non-transitory recording medium, such as a memory card, in which the program has been stored.

As shown in FIG. 1, the processing member 50 includes an acquirer 53, a learning member 54, and an output 56 in addition to the assessing member 55. In FIG. 1, the acquirer 53, the learning member 54, the assessing member 55, and the output 56 are not tangible components but are functions implemented by the processing member 50.

The acquirer 53 acquires the detection result output from the sensor device which is the detector 1.

The learning member 54 causes a program (algorithm) of artificial intelligence to create the learned model by machine learning of the learning data and causes the storage 52 to store the learned model. That is, the learning member 54 is in charge of a learning phase in which in a preparation stage before the ripeness of fruit whose ripeness is unknown is assessed by using the ripeness assessing system 5, combinations of detection results and ripeness of fruit whose ripeness is known are accumulated as learning data in the storage 52, and from the learning data, a learned model MD1 is created. Note that after the learned model MD1 is created, the learning member 54 may perform learning again by using learning data newly collected by the acquirer 53 to improve the performance of the learned model MD1.

The assessing member 55 uses the learned model MD1 stored in the storage 52 to assess, based on a detection result, the ripeness of fruit. Assessing the ripeness of fruit means assessing the degree of ripening by a method which allows humans to recognize the degree of ripening. For example, the assessing member 55 may assess the ripeness of fruit by selecting a numerical value corresponding to the degree of ripening of the fruit, may assess the ripeness of fruit by selecting a color corresponding to the degree of ripening of the fruit, or may assess the ripeness of fruit by selecting wording corresponding to the degree of ripening of the fruit.

The output 56 outputs a result of assessing the ripeness of the fruit by the assessing member 55 to the display member 57.

The storage 52 includes one or more storage devices. The storage device is, for example, RAM, ROM, or EEPROM. The storage 52 stores the learned model MD1, and the like. The learned model MD1 may be created in the learning phase using the ripeness assessing system 5 as described above or may be created by a learning system other than the ripeness assessing system 5. When the learned model MD1 is created by a learning system other than the ripeness assessing system 5, the ripeness assessing system 5 does not have to include the learning member 54.

The display member 57 externally displays the result of the assessing output from the output 56 by a method which allows humans to recognize the result of the assessing. The display member 57 is, for example, a device that visibly displays a result of assessing the ripeness of fruit, and in that case, the display member 57 includes, for example, a display device such as a liquid crystal display. The display member 57 may be a device that presents a result of assessing the ripeness of fruit by using voice, and in that case, the display member 57 includes, for example, a buzzer or a loudspeaker.

An example of operation of the ripeness assessing system 5 in an inference phase will be described. Note that the operation in the inference phase is a mere example as explained above, and the order of processes may accordingly be changed, or a process(es) may accordingly be added or omitted.

A user gives an operation to, for example, a power supply switch to activate the processing member 50, thereby causing the ripeness assessing system 5 to start the operation of assessing the ripeness of fruit. The user introduces the volatile component released from the fruit into the accommodation space 11 through the introduction path 12, thereby exposing the gas sensor 2 to the volatile component (exposure step).

The acquirer 53 acquires a detection result which is, for example, the electrical resistance value of the gas sensor 2 (acquisition step ST3). The assessing member 55 inputs the detection result acquired by the acquirer 53 to the learned model MD1, thereby assessing the ripeness (assessment step).

When the ripeness is assessed by the assessing member 55, the output 56 outputs an assessment result by the assessing member 55 to the display member 57 (output step). Thus, the user checks contents displayed by the display member 57, thereby checking the ripeness of the fruit.

The embodiment described above is a mere example of various embodiments of the present disclosure. The embodiment described above may be modified variously depending on design and the like as long as the object of the present disclosure is achieved. Variations of the embodiment will be enumerated below. Any of the variations to be described below may be combined as appropriate.

Fruit whose ripeness is to be assessed may have been, but does not have to have been, afterripened. If the fruit has not been afterripened, the ripeness of the fruit is difficult to be externally grasped, but even in such a case, the present embodiment enables the ripeness of the fruit to be assessed.

The method for assessing ripeness according to the present embodiment may include assessing, based on the result of assessing the ripeness, an afterripening condition for the fruit. The afterripening condition for the fruit includes, for example, at least one selected from the group consisting of: the temperature and humidity in an atmosphere such as a room; the composition of a gas in the atmosphere (e.g., the concentration of each of ethylene and carbon dioxide in the atmosphere); a time period required for the afterripening; and the like. For example, the afterripening condition is assessed, based on the ripeness required when the fruit is placed on sale in a retail establishment and a requested delivery date of the fruit by a retailer, such that the ripeness of the fruit requested by the retail establishment is achieved on the delivery date. In this case, it becomes easy to place the fruit on sale after the fruit is appropriately ripened through the afterripening. The afterripening condition for the fruit is assessed, for example, by creating, in advance, a database including correspondence relationships between ripeness and afterripening conditions accumulated therein, searching for a ripening condition corresponding to ripeness in the database, and outputting a searched result. Moreover, similarly to the case of the assessment of the ripeness, to assess the afterripening condition, for example, combinations of assessment results of ripeness and afterripening conditions may be accumulated as learning data in advance, the learning data may be used to create a learned model for assessing an afterripening condition from an assessment result of ripeness, and the learned model may be used to assess an afterripening condition from an assessment result of the ripeness of fruit for which an afterripening condition is unknown.

The ripeness assessing system 5 in the present disclosure includes a computer system in, for example, the processing member 50. The computer system includes a processor and memory as principal hardware components thereof. The processor executes a program stored in the memory of the computer system, thereby implementing a function as the ripeness assessing system 5 in the present disclosure. The program may be stored in advance in the memory of the computer system. Alternatively, the program may also be downloaded through a telecommunications network or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

Also, the plurality of functions of the ripeness assessing system 5 are aggregated together in a single housing. However, this is not an essential configuration for the ripeness assessing system 5. Alternatively, those constituent elements of the ripeness assessing system 5 may be distributed in multiple different housings. Further, at least some functions of the ripeness assessing system 5 (e.g., some functions of the ripeness assessing system 5) may be implemented as a cloud computing system, for example.

In the ripeness assessing system 5 of the embodiment described above, the gas sensor 2 includes 16 sensor elements Ax, but the number of sensor elements Ax may accordingly be changed. Moreover, in the ripeness assessing system 5 of the embodiment described above, the 16 sensor elements Ax are arranged in four rows and four columns, but the arrangement of the plurality of sensor elements Ax is not limited to that in the embodiment described above. The plurality of sensing elements may be arranged linearly or may be arranged in one or more concentric circles with a space provided therebetween.

In the ripeness assessing system 5 of the embodiment described above, the learned model MD1 is stored in the storage 52 of the ripeness assessing system 5, but the ripeness assessing system 5 may use the learned model MD1 on the cloud to assess the ripeness of fruit. That is, the assessing member 55 of the ripeness assessing system 5 may input the detection result output from the detector 1 to the learned model on the cloud to acquire an assessment result from the learned model on the cloud, thereby assessing the ripeness of fruit. Examples

As an example, a result of verifying that the method for assessing ripeness of fruit and the system for assessing ripeness according to the present embodiment can assess the ripeness of fruit is shown below.

### 1. Assessment of Ripeness of Bananas

### (1) Measurement by Using Gas Chromatograph

At the Osaka Municipal Central Wholesale Market, bananas from the Philippines which were green and non-afterripened were purchased, and the bananas were immediately placed in a room at 23° C. On each of days 1, 4, 7, 11, 14, 17, 22, 25, 29, 32, and 37 from the day on which the bananas were placed in the room, a gas (3L) including a volatile component released from an epicarp of each banana was introduced into an adsorption tube having an adsorption agent (tenax) put therein, was adsorbed on the adsorption agent, and was thereby collected. The adsorption tube was heated to cause the adsorption agent to desorb the gas, and the gas was analyzed with a gas chromatograph mass spectrometer (manufactured by Shimadzu Corporation, GCMS-QP2010 Ultra) under the following condition.

### a Configuration of Gas Chromatograph

Column: InertCap 5MS/Sil. Inner diameter 0.25 mm, length 30 m, film thickness 0.25 μm, guard column 10 m.
Inlet port: OPTIC4.
Autosampler: AOC-5000.

### b Supply Condition of Gas to Column

Heating temperature of adsorption tube: The temperature of the adsorption tube was increased from 35° C. to 250° C. at a rate of temperature rise of 20° C./sec, and the adsorption tube was maintained at 250° C. for 3 minutes.
Injection mode: Splitless.
Carrier gas: Helium.
Flow rate: 1 ml/min.
Cryo Trap: −100° C. (3 minutes).
Column heating condition: The column was maintained at 30° C. for 10 minutes, the temperature of the column was subsequently increased to 100° C. at a rate of temperature rise of 2° C./min, was subsequently increased to 200° C. at a rate of temperature rise of 4° C./min, was subsequently increased to 250° C. at a rate of temperature rise of 10° C./min, and was subsequently kept at 250° C. for 5 minutes.

### c Mass Spectrometry Condition

Ionization: EI.
Ion source temperature: 200° C.
m/z range: m/z35-300(Scan mode).
Interface temperature: 200° C.

### (2) Measurement by Using Sensor Device

A sensor device was prepared which includes gas sensors (sensor array) including 16 sensor elements including respective organic materials which are adipic acid polydiethylene glycol, succinic acid diethylene glycol, diglycerol, tetrahydroxy ethylene diamine, poly(ethylene glycol succinate), polyethylene glycol 4000 (manufactured by Sigma-Aldrich Co. LLC), polyethylene glycol 20000 (manufactured by Sigma-Aldrich Co. LLC), polyethylene glycol 20M (manufactured by Shinwa Chemical Industries Ltd.), free fatty acid phase (Free Fatty Acid Polymer, manufactured by Shinwa Chemical Industries Ltd.), 1,2,3-tris (2-cyanoethoxy) propane, N,N-bis(2-cyanoethyl) formamide, Lac-3R-728 (manufactured by GL Sciences Inc.), Reoplex 400 (manufactured by Shinwa Chemical Industries Ltd.), SP-2330 (manufactured by Sigma-Aldrich Co. LLC), SP-2340 (manufactured by Sigma-Aldrich Co. LLC), and UCON 75-HB-90000 (manufactured by Shinwa Chemical Industries Ltd.).

For the same bananas as that used in “(1) Measurement by Using Gas Chromatograph” described above, a volatile component released from the epicarp of each banana was collected on each of days 1, 4, 7, 11, 14, 17, 22, 25, 29, 32, and 37 from the day on which the bananas were placed in the room. While a voltage was applied to each sensor element of the sensor device, each sensor element was exposed to the volatile component for 9 seconds, and then, the gas sensor was exposed to clean air for 27 seconds. This process was repeated five times. A collection of changes in a current flowing through each sensor element in this period was acquired as a detection result.

### (3) Verification of Ripeness of Fruit

For the same bananas as that used in “(1) Measurement by Using Gas Chromatograph” described above, an image of a surface of the epicarp of each banana was captured on each of days 1, 4, 7, 11, 14, 17, 22, 25, 29, 32, and 37 from the day on which the bananas were placed in the room, and based on images thus obtained, a hue (HUE value), in the range from 0° to 360°, of the epicarp of each banana was measured. Note that the closer the HUE is to 0°, the more reddish the color of the epicarp is, and the closer the HUE is to 60°, the more yellowish the color of the epicarp is. Here, the color of the surface of the epicarp is not uniform, and therefore, an average value of HUE values of the epicarp in the images were calculated by using an ImageJ which is image processing software, and the average value is defined as the HUE value of the epicarp. On each of the days described above, a HUE value of the epicarp of each of the plurality of bananas was measured. Moreover, the exterior of the epicarp was visually checked, and at the start of a test, the color of the epicarp was strongly greenish but was yellowish around day 11, was generally yellow around day 22, was blackish around day 29, and was generally black on day 37.

Moreover, for the same bananas as that used in “(1) Measurement by Using Gas Chromatograph” described above, the epicarps of the bananas were removed on each of days 1, 4, 7, 11, 14, 17, 22, 25, 29, 32, and 37 from the day on which the bananas were placed in the room, and a V-shaped probe (A-5) of a force gauge (item number DS2-50N manufactured by IMADA Co., Ltd.) was pushed in a mesocarp of each banana in a peak value measurement mode so that a root of the probe sank in the mesocarp. A measured value by the force gauge at that time was recorded. The same measurement was performed seven times, a maximum value and a minimum value were excluded from measured values thus obtained, and an average value of the remaining measured values was obtained. The average value was defined as an index of hardness of the mesocarp.

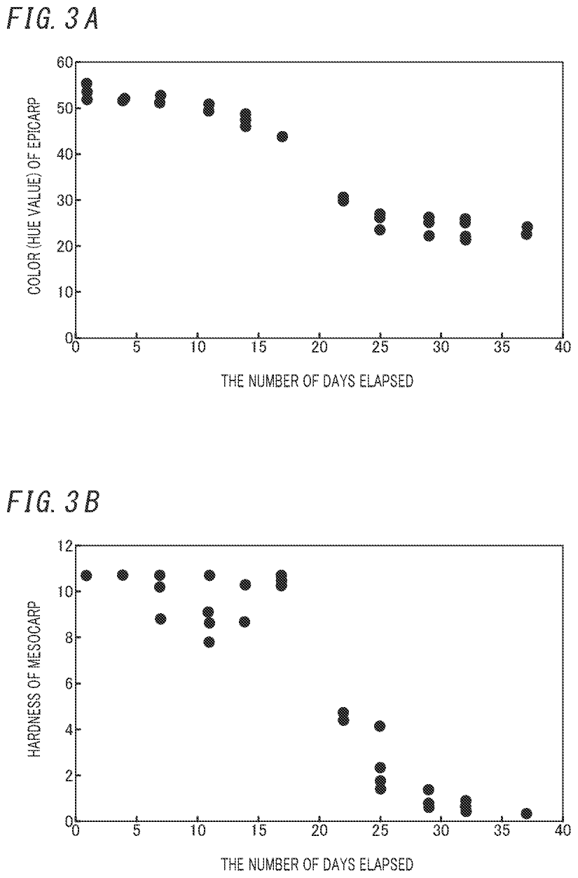

The graph in FIG. 3A shows a relationship between the number of days elapsed since the start of the test and the color (HUE value) of the epicarp. In FIG. 3A, the horizontal axis represents the number of days elapsed since the start of the test, and the vertical axis represents the HUE value of the epicarp. Moreover, the graph in FIG. 3B shows a relationship between the number of days elapsed since the start of the test and the hardness (value measured with the force gauge) of the mesocarp. In FIG. 3B, the horizontal axis represents the number of days elapsed since the start of the test, and the vertical axis represents the value (unit N) of the mesocarp measured with the force gauge.

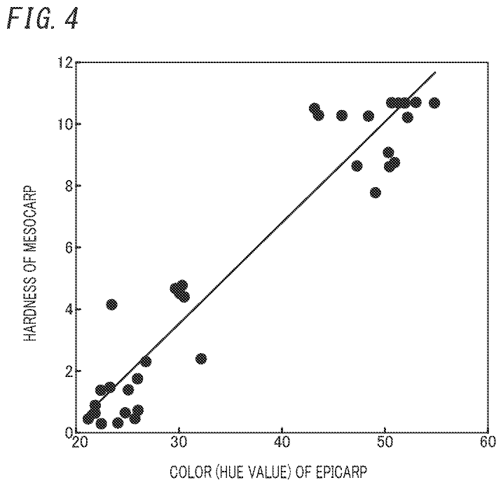

A change in the color of the epicarp and a change in the hardness of the mesocarp with the passage of days are similar to each other. FIG. 4 shows a relationship between the color of the epicarp and the hardness of the mesocarp. According to FIG. 4, a correlation between the color of the epicarp and the hardness of the mesocarp was high, and when a determination coefficient (R²) between the color of the epicarp and the hardness of the mesocarp was calculated, a value as high as 0.93 was obtained.

Thus, both the color of the epicarp and the hardness of the mesocarp can similarly be determined to be ripeness indices for bananas. That is, both assessing the color of the epicarp of a banana and assessing the hardness of the mesocarp of a banana can be synonymous with assessing the ripeness of a banana.

### (4) Verification of Use of Gas Chromatograph as Detector

Combinations of chromatograms which are detection results obtained by “(1) Measurement by Using Gas Chromatograph” described above and colors of the epicarps of the bananas were accumulated as learning data, and from the learning data, a learned model was created by the random forest method, the learned model being an algorithm for assessing, from a detection result of a banana, the color of the epicarp of the banana. Note that to create the learned model, 80% of the learning data was used as teaching data to configure a classifier, and the remaining 20% of the learning data was used for test data.

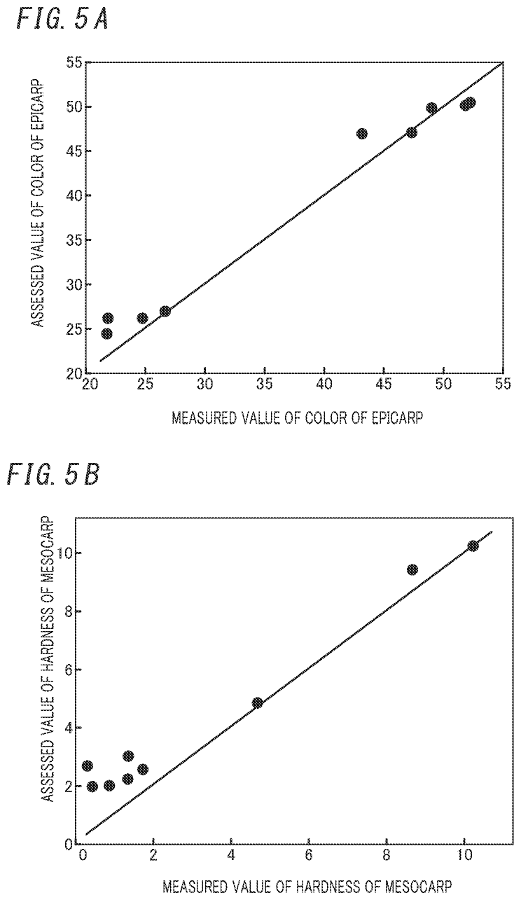

The graph in FIG. 5A shows a relationship between a result of assessing the color of the epicarp of the banana from the detection results included in the test data by using the learned model and an actual color, included in the test data, of the epicarp of the banana. The vertical axis of the graph represents the result of assessing the color of the epicarp of the banana, and the horizontal axis represents the actual color of the epicarp of the banana. As this graph shows, a correlation between the assessment result and an actual value was high, and when a determination coefficient (R²) between the result of assessing and the actual value was calculated, a value as high as 0.967 was obtained, and an average absolute error was a value as low as 1.847.

Moreover, combinations of the chromatograms which are detection results obtained by “(1) Measurement by Using Gas Chromatograph” described above and hardness of the mesocarps of the bananas were accumulated as learning data, and from the learning data, a learned model was created by the random forest method, the learned model being an algorithm for assessing, from a detection result of a banana, the hardness of the mesocarp of the banana. Note that to create the learned model, 80% of the learning data was used as teaching data to configure a classifier, and the remaining 20% of the learning data was used for test data.

The graph of FIG. 5B shows a relationship between a result of assessing the hardness of the mesocarp of the banana from the detection results included in the test data by using the learned model and actual hardness, included in the test data, of the mesocarp of the banana. The vertical axis of this graph represents the result of assessing the hardness of the mesocarp of the banana, and the horizontal axis represents the actual hardness of the mesocarp of the banana. As this graph shows, a correlation between the assessment result and an actual value was high, and when a determination coefficient (R²) between the result of assessing and the actual value was calculated, a value as high as 0.877 was obtained, and an average absolute error was a value as low as 1.026.

From the above description it can be understand that the ripeness of the banana can be relatively accurately assessed based on the detection result obtainable by using the chromatograph as the detector.

Moreover, the above description suggests that in the learned model for assessing the color of the epicarp of the banana and the learned model for assessing the hardness of the mesocarp of the banana, respective signals derived from 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate are involved in the assessment.

In particular, in the learned model for assessing the color of the epicarp of the banana, the significance of contribution, to the assessment, of the respective signals derived from isopentyl acetate, 1-methyl hexyl butyrate, 2-pentanone, 2-pentanol acetate, isobutyl isovalerate, isopentyl acetate, and isoamyl butyrate included in the volatile component decreases in this order.

Moreover, in the learned model for assessing the hardness of the mesocarp of the banana, the significance of contribution, to the assessment, of the respective signals derived from 2-methoxyfuran, isobutyl alcohol, 1-methyl hexyl butyrate, isoamyl butyrate, isopentyl acetate, and butyl butyrate included in the volatile component decreases in this order.

Thus, the marker component preferably includes at least one component selected from the group consisting of, in particular, 1-methyl hexyl butyrate, 2-methoxyfuran, 2-pentanol acetate, 2-pentanone, butyl butyrate, isoamyl butyrate, isobutyl alcohol, isopentyl acetate, and isobutyl isovalerate.

### (5) Verification of Use of Sensor Device as Detector

Combinations of detection results obtained by “(2) Measurement by Using Sensor Device” described above and colors of the epicarps of the bananas were accumulated as learning data, and from the learning data, a learned model was created, the learned model being an algorithm for assessing, from a detection result of a banana, the color of the epicarp of the banana by the neural network. Note that to create the learned model, 80% of the learning data was used as teaching data to configure a classifier, and the remaining 20% of the learning data was used for test data.

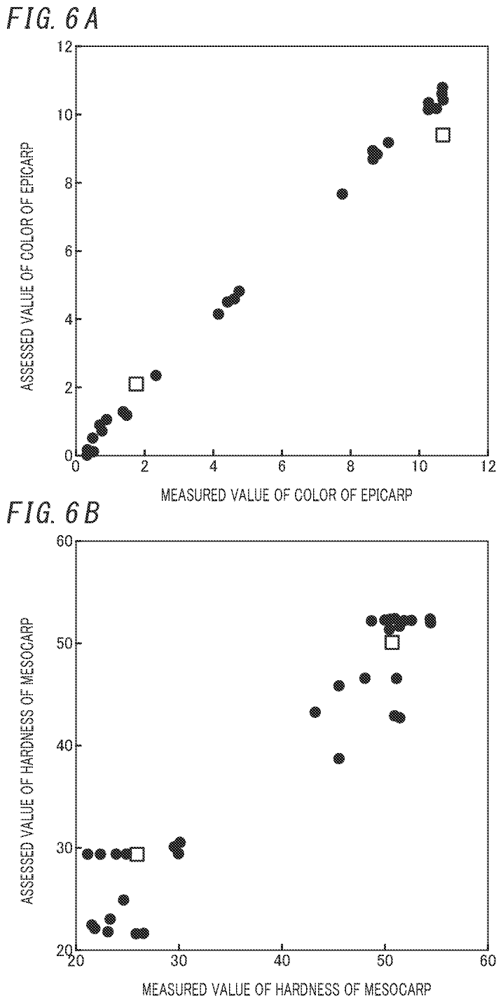

The graph in FIG. 6A shows a relationship between a result of assessing the color of the epicarp of the banana from the detection results included in the test data by using the learned model and an actual color, included in the test data, of the epicarp of the banana. The vertical axis of the graph represents the result of assessing the color of the epicarp of the banana, and the horizontal axis represents the actual color of the epicarp of the banana. As this graph shows, a correlation between the assessment result and an actual value was high, and when a determination coefficient (R²) between the result of assessing and the actual value was calculated, a value as high as 0.88 was obtained, and a root mean square error (RMSE) was a value as low as 7.7.

Moreover, combinations of the detection results obtained by “(2) Measurement by Using Sensor Device” described above and hardness of the mesocarps of the bananas were accumulated as learning data, and from the learning data, a learned model was created, the learned model being an algorithm for assessing, from a detection result of a banana, the hardness of the mesocarp of the banana by the neural network. Note that to create the learned model, 80% of the learning data was used as teaching data to configure a classifier, and the remaining 20% of the learning data was used for test data.

The graph in FIG. 6B shows a relationship between a result of assessing the hardness of the mesocarp of the banana from the detection results included in the test data by using the learned model and actual hardness, included in the test data, of the mesocarp of the banana. The vertical axis of this graph represents the result of assessing the hardness of the mesocarp of the banana, and the horizontal axis represents the actual hardness of the mesocarp of the banana. As this graph shows, a correlation between the assessment result and an actual value was high, and when a determination coefficient (R²) between the result of assessing and the actual value was calculated, a value as high as 0.99 was obtained, and the root mean square error (RMSE) was a value as low as 2.2.

From the above description it can be understand that the ripeness of the banana can be relatively accurately assessed based on the detection result obtainable by using, as the detector, the sensor device including the gas sensor.

Moreover, according to the results above, it can be determined that both when the gas chromatograph is used as the detector and when the sensor device is used as the detector, the ripeness of the banana can be assessed in a wide time period from a state where the color of the epicarp of the banana is generally greenish until the epicarp becomes generally black.

### (6) References

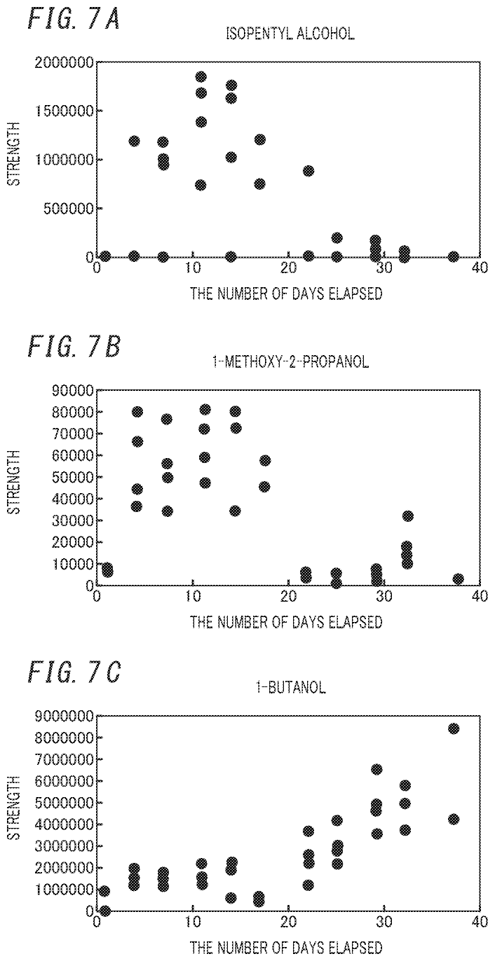

For reference, FIGS. 7A to 7C show relationships of the number of days elapsed to the strengths of peaks respectively corresponding to isopentyl alcohol, 1-methoxy-2-propanol, and 1-butanol and appearing in the chromatograms which are the detection results obtained by “(1) Measurement by Using Gas Chromatograph”. FIG. 7A is a result regarding isopentyl alcohol. FIG. 7B is a result regarding 1-methoxy-2-propanol. FIG. 7C is a result regarding 1-butanol. As these figures show, changes in the amounts of isopentyl alcohol, 1-methoxy-2-propanol, and 1-butanol in accordance with the number of days elapsed are different from each other. It is inferred that a compound including isopentyl alcohol, a compound including 1-methoxy-2-propanol, and a compound including 1-butanol which can be marker components are produced through the metabolism of bananas, but as FIGS. 7A to 7C show, the discharge amounts of these compounds may increase or decrease in the ripening process of the bananas. This presumably reflects that in the ripening process of the bananas, these compounds undergo chemical reaction, such as production, polymerization, and decomposition in the bananas. Therefore, using these compounds as marker components presumably enables the ripeness of the bananas to be assessed with good accuracy.

### 2. Assessment of Ripeness of Avocados

### (1) Measurement by Using Gas Chromatograph

In a retail establishment, avocados (Hass) were purchased, and the avocados were immediately placed in a room at 23° C. On each of days 0, 1, 4, and 7 from the day on which the avocados were placed in the room, a gas (3L) including a volatile component released from an epicarp of each avocado was introduced into an adsorption tube having an adsorption agent (tenax) put therein, was adsorbed on the adsorption agent, and was thereby collected. The adsorption tube was heated to cause the adsorption agent to desorb the gas, and the gas was analyzed with a gas chromatograph mass spectrometer (manufactured by Shimadzu Corporation, GCMS-QP2010 Ultra) under a condition. The condition is the same as that in “1. Assessment of Ripeness of Bananas” described above.

### (2) Measurement by Using Sensor Device

The same sensor device as that in “1. Assessment of Ripeness of Bananas” described above was prepared.

For the same avocados as that used in “(1) Measurement by Using Gas Chromatograph” described above, a volatile component released from the epicarp of each avocado was collected on each of days 0, 1, 4, and 7 from the day on which the avocados were placed in the room. While a voltage was applied to each sensor element of the sensor device, each sensor element was exposed to the volatile component for 9 seconds, and then, the gas sensor was exposed to clean air for 27 seconds. This process was repeated five times. A collection of changes in a current flowing through each sensor element in this period was acquired as a detection result.

### (3) Verification of Ripeness of Fruit

For the same avocados as that used in “(1) Measurement by Using Gas Chromatograph” described above, the epicarps of the avocados were removed, and an image of the surface of a mesocarp was captured on each of days 0, 1, 4, and 7 from the day on which the avocados were placed in the room, and based on images thus obtained, a hue (HUE value), in the range from 0° to 360°, of the mesocarp of each avocado was measured. Here, the color of the surface of the mesocarp is not uniform, and therefore, an average value of HUE values of the mesocarp in the images were calculated by using an ImageJ which is image processing software, and the average value is defined as the HUE value of the mesocarp. On each of the days described above, a HUE value of the mesocarp of each of the plurality of avocados was measured. Moreover, the exterior of the mesocarp was visually checked, and at the start of a test, the color of the mesocarp was generally greenish and gradually became yellowish. Note that almost no change in the color of the epicarp was observed.

Moreover, for the same avocados as that used in “(1) Measurement by Using Gas Chromatograph” described above, the epicarps of the avocados were removed on each of days 0, 1, 4, and 7 from the day on which the avocados were placed in the room, and an M-shaped probe of a force gauge (item number DS2-50N manufactured by IMADA Co., Ltd.) was pushed in the mesocarp of each avocado in a peak value measurement mode so that a root of the probe sank in the mesocarp. A measured value by the force gauge at that time was recorded. The same measurement was performed seven times, a maximum value and a minimum value were excluded from measured values thus obtained, and an average value of the remaining measured values was obtained. The average value was defined as an index of hardness of the mesocarp.

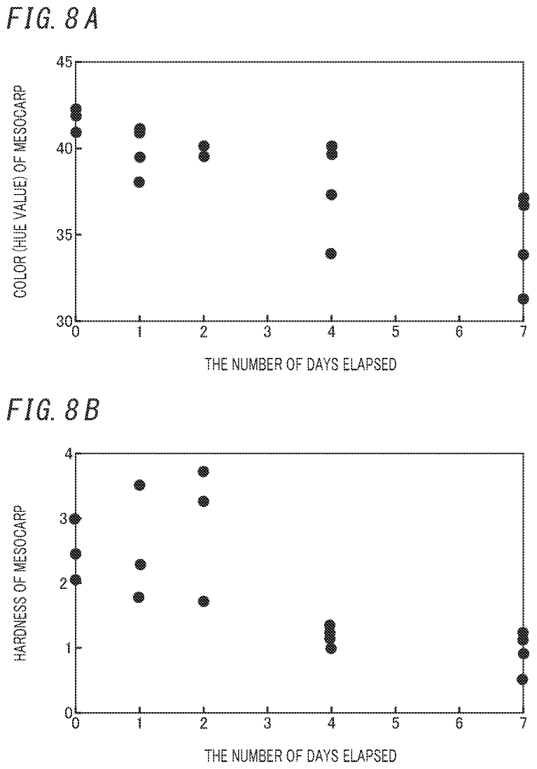

The graph in FIG. 8A shows a relationship between the number of days elapsed since the start of the test and the color (HUE value) of the mesocarp. In FIG. 8A, the horizontal axis is the number of days elapsed since the start of the test, and the vertical axis is the HUE value of the mesocarp. Moreover, the graph in FIG. 8B shows a relationship between the number of days elapsed since the start of the test and the hardness (value measured with the force gauge) of the mesocarp. In FIG. 8B, the horizontal axis represents the number of days elapsed since the start of the test, and the vertical axis represents the value (unit N) of the mesocarp measured with the force gauge.

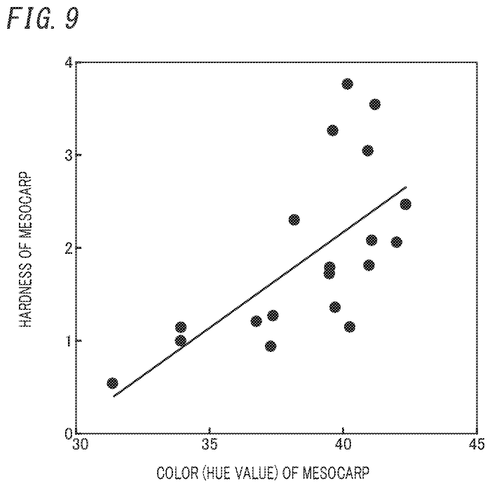

A change in the color of the mesocarp and a change in the hardness of the mesocarp with the passage of days are similar to each other. FIG. 9 shows a relationship between the color of the mesocarp and the hardness of the mesocarp. According to FIG. 9, a correlation between the color of the mesocarp and the hardness of the mesocarp was high, and when a determination coefficient (R²) between the color of the mesocarp and the hardness of the mesocarp was calculated, a value as high as 0.42 was obtained.

Therefore, the color of the mesocarp and the hardness of the mesocarp can be determined, in a similar manner, to be ripeness indices for avocados. That is, both assessing the color of the mesocarp of an avocado and assessing the hardness of the mesocarp of an avocado can be synonymous with assessing the ripeness of an avocado.

### (4) Verification of Use of Gas Chromatograph as Detector

Combinations of chromatograms which are detection results obtained by “(1) Measurement by Using Gas Chromatograph” described above and colors of the mesocarps of the avocados were accumulated as learning data, and from the learning data, a learned model was created by the random forest method, the learned model being an algorithm for assessing, from a detection result of an avocado, the color of the mesocarp of the avocado. Note that to create the learned model, 80% of the learning data was used as teaching data to configure a classifier, and the remaining 20% of the learning data was used for test data.

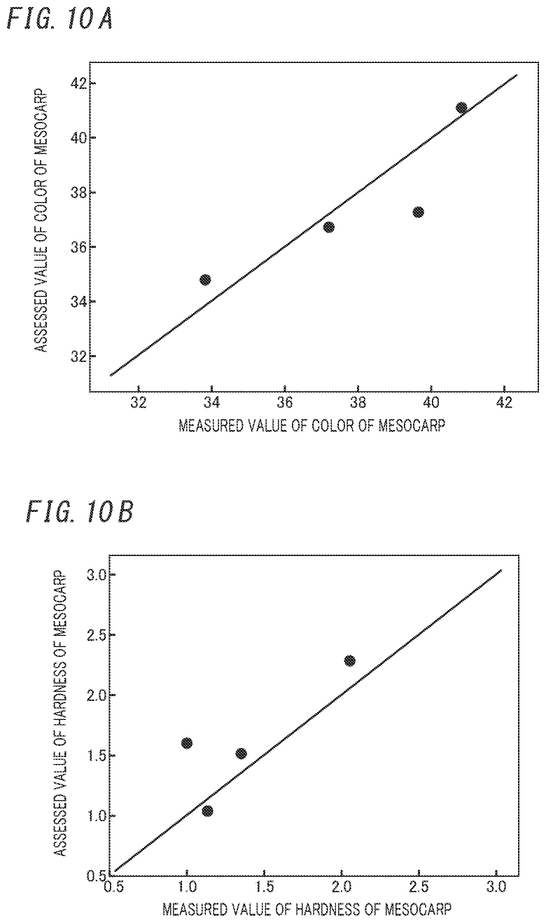

The graph in FIG. 10A shows a relationship between a result of assessing the color of the mesocarp of the avocado from the detection results included in the test data by using the learned model and an actual color, included in the test data, of the mesocarp of the avocado. The vertical axis of the graph represents the result of assessing the color of the mesocarp of the avocado, and the horizontal axis represents the actual color of the mesocarp of the avocado. As this graph shows, a correlation between the assessment result and an actual value was high, and when a determination coefficient (R²) between the result of assessing and the actual value was calculated, a value as high as 0.774 was obtained, and an average absolute error was a value as low as 0.973.

Moreover, combinations of the chromatograms which are the detection results obtained by “(1) Measurement by Using Gas Chromatograph” described above and hardness of the mesocarps of the avocados were accumulated as learning data, and from the learning data, a learned model was created by the random forest method, the learned model being an algorithm for assessing, from a detection result of an avocado, the hardness of the mesocarp of the avocado. Note that to create the learned model, 80% of the learning data was used as teaching data to configure a classifier, and the remaining 20% of the learning data was used for test data.

The graph in FIG. 10B shows a relationship between a result of assessing the hardness of the mesocarp of the avocado from the detection results included in the test data by using the learned model and actual hardness, included in the test data, of the mesocarp of the avocado. The vertical axis of the graph represents the result of assessing the hardness of the mesocarp of the avocado, and the horizontal axis represents the actual hardness of the mesocarp of the avocado. As this graph shows, a correlation between the assessment result and an actual value was high, and when a determination coefficient (R²) between the result of assessing and the actual value was calculated, a value as high as 0.325 was obtained, and an average absolute error was a value as low as 0.272.

Therefore, it can be understood that based on the detection result obtained by using the chromatogram as the detector, the ripeness of the avocado can be relatively accurately assessed.

Moreover, the above description suggests that in the learned model for assessing the color of the mesocarp of the avocado and the learned model for assessing the hardness of the mesocarp of the avocado, respective signals derived from 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone are involved in the assessment.

In particular, in the learned model for assessing the color of the mesocarp of the avocado, the significance of contribution, to the assessment, of the respective signals derived from 3-(1-methyl ethenyl)toluene, xylene, 2-propanol, 2-octanone, and 4-ethoxy-2-butanone included in the volatile component decreases in this order.

Moreover, in the learned model for assessing the hardness of the mesocarp of the avocado, the significance of contributions, to the assessment, of the respective signals derived from 2,3,6,7-tetra methyl octane, menthol, 2,2,8-trimethyldecane, and limonene included in the volatile component decreases in this order.

### (5) Verification of Use of Sensor Device as Detector

Combinations of detection results obtained by “(2) Measurement by Using Sensor Device” described above and colors of the mesocarps of the avocados were accumulated as learning data, and from the learning data, a learned model was created, the learned model being an algorithm for assessing, from a detection result of an avocado, the color of the mesocarp of the avocado by the neural network. Note that to create the learned model, 80% of the learning data was used as teaching data to configure a classifier, and the remaining 20% of the learning data was used for test data.

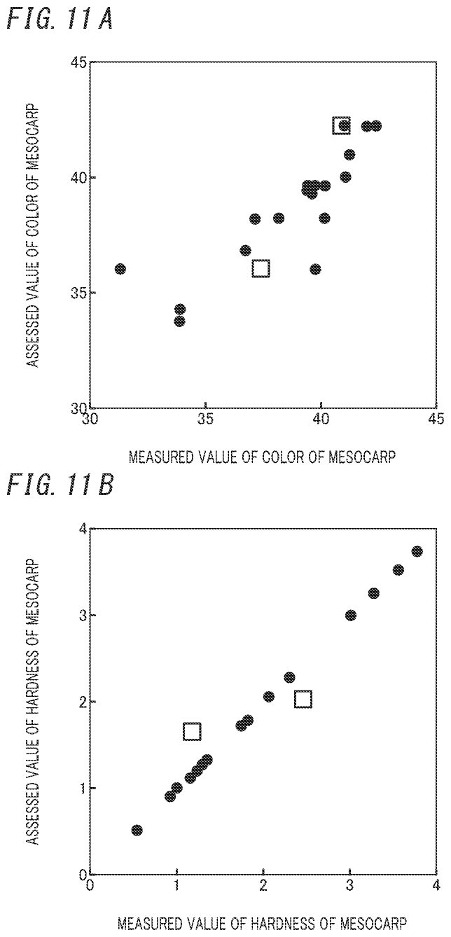

The graph in FIG. 11A shows a relationship between a result of assessing the color of the mesocarp of the avocado from the detection results included in the test data by using the learned model and an actual color, included in the test data, of the mesocarp of the avocado. The vertical axis of the graph represents the result of assessing the color of the mesocarp of the avocado, and the horizontal axis represents the actual color of the mesocarp of the avocado. As this graph shows, a correlation between the assessment result and an actual value was high, and when a determination coefficient (R²) between the result of assessing and the actual value was calculated, a value as high as 0.71 was obtained

Moreover, combinations of the detection results obtained by “(2) Measurement by Using Sensor Device” described above and hardness of the mesocarps of the avocados were accumulated as learning data, and from the learning data, a learned model was created, the learned model being an algorithm for assessing, from a detection result of an avocado, the hardness of the mesocarp of the avocado by the neural network. Note that to create the learned model, 80% of the learning data was used as teaching data to configure a classifier, and the remaining 20% of the learning data was used for test data.

The graph in FIG. 11B shows a relationship between a result of assessing the hardness of the mesocarp of the avocado from the detection results included in the test data by using the learned model and actual hardness, included in the test data, of the mesocarp of the avocado. The vertical axis of the graph represents the result of assessing the hardness of the mesocarp of the avocado, and the horizontal axis represents the actual hardness of the mesocarp of the avocado. As this graph shows, a correlation between the assessment result and an actual value was high, and when a determination coefficient (R²) between the result of assessing and the actual value was calculated, a value as high as 0.97 was obtained.

From the above description it can be understand that the ripeness of the avocado can be relatively accurately assessed based on the detection result obtainable by using, as the detector, the sensor device including the gas sensor.

Moreover, when the sensor device including the gas sensor is used as the detector, the correlation between the assessment result and the actual value is higher than when the gas chromatograph is used as the detector. This has the possibility that a column of the gas chromatograph has selectivity and can thus not cover all compounds in the volatile component in one measurement, whereas in the sensor device, the gas sensor recognizes a variety of types of compounds, thereby contributing to an improvement in the accuracy of the assessment. Note that the measurement using the gas chromatograph in this case detects no compound, such as methane and ethane, having a low boiling point.

Note that 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone which are marker components in this case are compounds produced through metabolism of avocados, and the discharge amounts of these compounds may increase or decrease in the ripening process of the avocados. This presumably reflects that in the ripening process of the avocados, these compounds undergo chemical reaction, such as production, polymerization, and decomposition in the avocados. Therefore, using these compounds as marker components presumably enables the ripeness of the avocados to be assessed with good accuracy.

As is clear from the embodiment and the example, the method for assessing ripeness of fruit according to a first aspect of the present disclosure is a fruit ripeness assessing method. The fruit is bananas. The method includes collecting a volatile component released from an epicarp of the fruit. The method includes acquiring, by using a detector (1), a detection result of the volatile component. The detector (1) is a means for outputting the detection result in accordance with an amount of one or more marker components selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate. The method includes assessing, based on the detection result, the ripeness of the fruit.

The first aspect enables the ripeness of a banana to be assessed without damaging the banana and readily enhances the accuracy of the assessment.

A method for assessing ripeness of fruit according to a second aspect of the present disclosure is a fruit ripeness assessing method. The fruit is avocados. The method includes collecting a volatile component released from an epicarp of the fruit. The method includes acquiring, by using a detector (1), a detection result of the volatile component. The detector (1) is a means for outputting the detection result in accordance with an amount of one or more marker components selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone. The method includes assessing, based on the detection result, the ripeness of the fruit.

The second aspect enables the ripeness of an avocado to be assessed without damaging the avocado and readily enhances the accuracy of the assessment.

In a third aspect of the present disclosure referring to the first or second aspect, the fruit is non-afterripened.

The third aspect enables the ripeness of the fruit to be assessed even in a state where the fruit is before afterripening and the ripeness of the fruit is thus difficult to be assessed from the exterior of the fruit.

A fourth aspect of the present disclosure referring to the third aspect further includes assessing, based on a result of the assessing of the ripeness, an afterripening condition for the fruit.

The fourth aspect enables the afterripening condition for the fruit to be assessed even in a state where the fruit is before afterripening and the ripeness of the fruit is thus difficult to be assessed from the exterior of the fruit.

In a fifth aspect of the present disclosure referring to any one of the first to fourth aspects, the detector (1) is a means for outputting the detection result according to an amount of each of two or more of the marker components.

The fifth aspect facilitates a further improvement in the accuracy of the assessment of the ripeness.

In a sixth aspect of the present disclosure referring to any one of the first to fifth aspects, the detector (1) includes a gas sensor (2).

The sixth aspect facilitates a further improvement in the accuracy of the assessment of the ripeness.

In a seventh aspect of the present disclosure referring to the sixth aspect, the gas sensor (2) is a sensor array including a plurality of sensor elements (Ax) having different sensory characteristics.

The seventh aspect facilitates a further improvement in the accuracy of the assessment of the ripeness.

In an eighth aspect of the present disclosure referring to any one of the first to fifth aspects, the detector (1) is a gas chromatograph.

The eighth aspect facilitates a further improvement in the accuracy of the assessment of the ripeness.

In a ninth aspect of the present disclosure referring to any one of the first to eighth aspects, the assessing is performed by executing an arithmetic process on the detection result.

The ninth aspect facilitates a further improvement in the accuracy of the assessment of the ripeness.

In a tenth aspect of the present disclosure referring to any one of the first to ninth aspects, the assessing is performed, based on the detection result, by using an evaluation model. The evaluation model is a learned model obtained by machine learning by using learning data.

The tenth aspect facilitates a further improvement in the accuracy of the assessment of the ripeness.

A ripeness assessing system (5) for fruit of an eleventh aspect of the present disclosure is a system for assessing ripeness of fruit, the system implementing the method for assessing the ripeness of the fruit of any one of the first to tenth aspects, the system including a detector (1) and an assessing member (55) configured to assess, based on the detection result output from the detector (1), the ripeness of the fruit.

The eleventh aspect enables the ripeness of bananas or avocados to be assessed without damaging the bananas or the avocados and facilitates an improvement in the accuracy of the assessment.

### Reference Signs List

1 Detector
2 Gas Sensor
5 Ripeness Assessing System
55 Assessing Member
Ax Sensor Element

1. A method for assessing ripeness of fruit, the fruit being bananas, the method comprising:
collecting a volatile component released from an epicarp of the fruit;
acquiring, by using a detector, a detection result of the volatile component, the detector being configured to output the detection result in accordance with an amount of one or more marker components selected from the group consisting of 1-butanol, 1-methyl hexyl butyrate, 1-methoxy-2-propanol, 2-methoxyfuran, 2-pentanol acetate, 2-pentanone, butyl butyrate, isoamyl butyrate, isobutyl alcohol, isopentyl alcohol (3-methyl-1-butanol), isopentyl acetate, isopentyl hexanoate, and isobutyl isovalerate; and
assessing, based on the detection result, the ripeness of the fruit.

2. A method for assessing ripeness of fruit, the fruit being avocados, the method comprising:
collecting a volatile component released from an epicarp of the fruit;
acquiring, by using a detector, a detection result of the volatile component, the detector being configured to output the detection result in accordance with an amount of one or more marker components selected from the group consisting of 2,3,6,7-tetra methyl octane, menthol, 2,2,8-trimethyldecane, limonene, 3-(1-methyl ethenyl)toluene, xylene, 2-propanol, 2-octanone, and 4-ethoxy-2-butanone; and
assessing, based on the detection result, the ripeness of the fruit.

3. The method of claim 1, wherein
the fruit is non-afterripened.

4. The method of claim 3, further comprising assessing, based on a result of the assessing of the ripeness, an afterripening condition for the fruit.

5. The method of claim 1, wherein
the detector is an output means for outputting the detection result according to an amount of each of two or more of the marker components.

6. The method of claim 1, wherein
the detector includes a gas sensor.

7. The method of claim 6, wherein
the gas sensor is a sensor array including a plurality of sensor elements having different sensory characteristics.

8. The method of claim 1, wherein
the detector is a gas chromatograph.

9. The method of claim 1, wherein
the assessing is performed by executing an arithmetic process on the detection result.

10. The method of claim 1, wherein
the assessing is performed, based on the detection result, by using an evaluation model, and
the evaluation model is a learned model obtained by machine learning by using learning data.

11. A system for assessing ripeness of fruit, the system implementing the method for assessing the ripeness of the fruit of claim 1, the system comprising:
the detector; and
an assessing member configured to assess, based on the detection result output from the detector, the ripeness of the fruit.

12. The method of claim 2, wherein
the fruit is non-afterripened.

13. The method of claim 12, further comprising assessing, based on a result of the assessing of the ripeness, an afterripening condition for the fruit.

14. The method of claim 2, wherein
the detector is an output means for outputting the detection result according to an amount of each of two or more of the marker components.

15. The method of claim 2, wherein
the detector includes a gas sensor.

16. The method of claim 15, wherein
the gas sensor is a sensor array including a plurality of sensor elements having different sensory characteristics.

17. The method of claim 2, wherein
the detector is a gas chromatograph.

18. The method of claim 2, wherein
the assessing is performed by executing an arithmetic process on the detection result.

19. The method of claim 2, wherein
the assessing is performed, based on the detection result, by using an evaluation model, and
the evaluation model is a learned model obtained by machine learning by using learning data.

20. A system for assessing ripeness of fruit, the system implementing the method for assessing the ripeness of the fruit of claim 2, the system comprising:
the detector; and
an assessing member configured to assess, based on the detection result output from the detector, the ripeness of the fruit.
